(19) United States
(12) Patent Application Publication
(10) Pub. No.: US 2006/0093643 A1
(43) Pub. Date: May 4, 2006

(54) Medical device for delivering therapeutic agents over different time periods

(76) Inventor: Eric B. Stenzel, Galway (IE)

Correspondence Address: JONES DAY, 222 EAST 41ST ST, NEW YORK, NY, 10017, US

(21) Appl. No.: 10/982,355

(22) Filed: Nov. 4, 2004

(51) Int. Cl. A61K 31/337; A61F 2/00

(52) U.S. Cl. 424/423; 514/449

(57) ABSTRACT

A medical device comprising a surface coated by at least two coating regions comprising a therapeutic agent is disclosed. These coating regions allow for the release of the therapeutic agent over different time periods.

### 1. FIELD OF THE INVENTION

The present invention relates generally to medical devices, such as stents, for delivering a therapeutic agent to a desired location within the body of a patient, such as a body lumen. More particularly, the medical device has a surface that is coated with at least two different coating regions that deliver a therapeutic agent to a patient over different time periods.

### 2. BACKGROUND OF THE INVENTION

A variety of medical conditions are commonly treated by introducing an insertable or implantable medical device into the body. In many instances, the medical device is coated with a material, such as a polymer, which is capable of releasing a therapeutic agent. For example, various types of drug-coated stents have been used for localized delivery of drugs to a body lumen. See, e.g., U.S. Pat. Nos. 6,099,562, 6,153,252 and 6,156,373.

Generally, the current coated medical devices release the therapeutic agent over a single time period. However, in some applications, it may be desirable to have the therapeutic agent released or delivered from the medical device coating over several different time periods. For instance, it may be desirable to have some of the therapeutic agent begin to release soon after the medical device is implanted and have some of the therapeutic agent begin to release at subsequent time(s). Therefore, there is a need for a medical device having a coating comprising a therapeutic agent in which the therapeutic agent is released over more than one time period, i.e., over different time periods.

### 3. SUMMARY OF THE INVENTION

The embodiments of the present invention related to medical devices, such as stents, that have a surface coated with at least two coating regions comprising a therapeutic agent. The therapeutic agent begins to release from the coating regions at different times, i.e.. the therapeutic agent from each coating region is released over different time periods.

In one embodiment, the medical device for delivering a therapeutic agent to a body tissue of a patient comprises a medical device having a surface. The medical device also comprises (a) a first coating region disposed on a first portion of the medical device surface, in which the first coating region comprises a first coating layer comprising a first therapeutic agent and (b) a second coating region disposed on a second portion of the medical device surface. The second coating region comprises a second coating layer comprising a second therapeutic agent; and at least a first additional coating layer disposed over the second coating layer. The first additional coating layer comprises a first biodegradable material and is capable of preventing the second therapeutic agent of the second coating layer from beginning to release from the second coating layer at the same time as the first therapeutic agent of the first coating layer begins to release from the first coating layer. The first coating region is capable of releasing the first therapeutic agent before the second coating region begins to release the second therapeutic agent. In certain embodiments, the second therapeutic agent begins to release before the release of the first therapeutic agent is completed. In other embodiments, the second therapeutic agent begins to release after the release of the first therapeutic agent is completed.

In certain embodiments, the first and second therapeutic agents can be the same. Also, in some embodiments, the first coating layer is not covered by any other coating layer. In addition, the first coating layer and the second coating layer can be contiguous. Moreover, the first additional coating layer can be disposed directly over the second coating layer or the first additional coating layer can be disposed indirectly over the second coating layer. In certain embodiments, the medical device can further comprise an intermediate coating layer disposed between the second coating layer and the first additional coating layer. The intermediate coating layer can comprise a biodegradable material and/or a third therapeutic agent. In some embodiments, the first coating layer further comprises a polymeric material and/or the second coating layer further comprises a polymeric material. In some embodiments, the first therapeutic agent can comprise an anti-thrombogenic agent, an anti-angiogenesis agent, an anti-proliferative agent, a growth factor, or a radiochemical. The anti-proliferative agent comprises paclitaxel, a paclitaxel analogue or a paclitaxel derivative.

In certain embodiments, the medical device can be a stent having a tubular sidewall in which the first portion of the medical device surface lies along a circumference of the tubular sidewall. In some embodiments, the medical device can be a stent having a tubular sidewall in which the first portion of the medical device surface lies along a longitudinal axis of the tubular sidewall.

In some embodiments, the medical device further comprises a third coating region disposed over a third portion of the medical device surface. The third coating region comprises a third coating layer comprising a third therapeutic agent; and at least a second additional coating layer disposed over the third coating layer. The second additional coating layer comprises a second biodegradable material. In some embodiments, the first, second and third therapeutic agent can be the same. In certain embodiments, the second additional coating layer can be disposed directly over the third coating layer or the second additional coating layer can be disposed indirectly over the third coating layer. Also, the medical device of claim can further comprise an intermediate coating layer disposed between the second coating layer and the first additional coating layer. The intermediate coating layer can comprise a biodegradable material and/or a fourth therapeutic agent. In certain embodiments, the first coating layer, is contiguous with at least the second coating layer or the third coating layer. Also, in some embodiments, the medical device further comprises at least a third additional coating layer disposed over the second additional coating layer. The third additional coating layer can comprise a third biodegradable material. In certain embodiments, the second additional coating layer further comprises a biologically active material.

In some embodiments, the first and second biodegradable materials can degrade at the same rate or the first and second biodegradable materials can degrade at different rates. In certain embodiments, the first additional coating layer can have a first thickness and the second additional coating layer can have a second thickness in which the first and second thicknesses are not the same. In other embodiments, the first and second thicknesses are the same. In some embodiments, the medical device is a stent having a tubular sidewall having an inner surface and an outer surface in which the first coating region and second coating region are disposed on the outer surface.

Moreover, in certain embodiments, the medical device further comprises a third coating region disposed on the inner surface. The third coating region comprises a third coating layer comprising a third therapeutic agent; and at least a second additional coating layer disposed over the third coating layer. The second additional coating layer can comprise a second biodegradable material.

In addition, in some embodiments, the medical device is a stent for delivering a therapeutic agent to patient which comprises a surface. The stent further comprises a first coating region disposed on a first portion of the surface. The first coating region comprises a first coating layer comprising a first therapeutic agent and a first polymeric material. Also, the stent comprises a second coating region disposed on a second portion of the medical device surface. The second coating region comprises a second coating layer comprising a second therapeutic agent and a second polymeric material; and at least an additional coating layer disposed over the second coating layer. The additional coating layer comprises a first biodegradable material. Also, the additional coating layer is capable of preventing the second therapeutic agent of the second coating layer from beginning to release from the second coating layer at the same time as the first therapeutic agent of the first coating layer begins to release from the first coating layer. The first coating region is capable of releasing the first therapeutic agent before the second coating region begins to release the second therapeutic agent. In certain embodiments, the second therapeutic agent begins to release before the release of the first therapeutic agent is completed. In other embodiments, the second therapeutic agent begins to release after the release of the first therapeutic agent is completed. Furthermore, the first coating layer and the second coating layer are contiguous and the additional coating layer is disposed directly over the second coating layer.

Moreover, in certain embodiments, the medical device is a stent for delivering a therapeutic agent to a patient comprising a tubular sidewall having an outer surface and an inner surface. The stent further comprises a first coating region disposed on a first portion of the outer surface, wherein the first coating region comprises a first coating layer comprising a first therapeutic agent and a polymeric material. The stent also comprises a second coating region disposed on a second portion of the outer surface. The second coating region comprises a second coating layer comprising a second therapeutic agent and the polymeric material; and at least a first additional coating layer disposed over the second coating layer. The first additional coating layer comprises a first biodegradable material and the first additional coating layer is capable of preventing the second therapeutic agent of the second coating layer from beginning to release from the second coating layer at the same time as the first therapeutic agent of the first coating layer begins to release from the first coating layer. The first coating region is capable of releasing the first therapeutic agent before the second coating region begins to release the second therapeutic agent. In certain embodiments, the second therapeutic agent begins to release before the release of the first therapeutic agent is completed. In other embodiments, the second therapeutic agent begins to release after the release of the first therapeutic agent is completed. The stent further comprises a third coating region disposed on a portion of the inner surface. The third coating region comprises a third coating layer comprising a third therapeutic agent and the polymeric material; as well as at least a second additional coating layer disposed over the third coating layer. The second additional coating layer comprises a second biodegradable material. Also, the first coating layer, and the second coating layer are contiguous and the first additional coating layer is disposed directly over the second coating layer. In some embodiments, the medical device comprises at least a third additional coating layer disposed over the second additional coating layer. In certain embodiments, third additional coating layer comprises a third biodegradable material. In addition to using biodegradable materials as the additional coating layers, bioabsorbable materials may also be used.

### 4. BRIEF DESCRIPTION OF THE DRAWINGS

FIG. 1 shows a side view of a stent suitable for use in the present invention.

FIGS. 2A-2E are cross-sectional views of embodiments of a coated medical device surface in accordance with the present invention.

FIGS. 3A-3C are cross-sectional views of other embodiments of a coated medical device surface of the present invention.

FIG. 4 is a cross-sectional view of an additional embodiment of a coated medical device surface of the present invention.

FIGS. 5A-5B show a stent having coating regions disposed along the longitudinal axis of the stent.

FIGS. 6A-6B show a stent having coating region disposed along the circumference of the stent.

### 5. DETAILED DESCRIPTION

The medical devices of the present invention comprise a medical device having a surface. Suitable medical devices include, but are not limited to, catheters, guide wires, balloons, filters (e.g., vena cava filters), stents, stent grafts, vascular grafts, intraluminal paving systems, implants and other devices. In certain embodiments, the medical devices are implanted or otherwise utilized in body lumina and organs such as the coronary vasculature, cranial vasculature, esophagus, trachea, colon, biliary tract, urinary tract, prostate, brain, and the like.

The filters that can be used in accordance with the present invention include, for example, thrombus filters that can be placed at a selected location within the vascular system and removed when no longer required. A preferred location for placement of these filters is the vena cava. Filters placed in the vascular system can intercept blood clots that may otherwise travel to the lungs and result in a pulmonary embolism, a life-threatening emergency that has become increasingly common. Further examples of filters that may be used in accordance with present invention include, e.g., those described in International Application No. WO 96/17634 and International Application No. WO 96/12448, both of which are herein incorporated by reference.

The grafts, including stent grafts, that may be used in accordance with the present invention include synthetic vascular grafts that can be used for replacement of blood vessels in part or in whole. A typical vascular graft is a synthetic tube with each end thereof sutured to the remaining ends of a blood vessel from which a diseased or otherwise damaged portion has been removed. In a typical stent graft, each end of the synthetic tube portion includes a stent that is affixed to each of the remaining ends of a blood vessel from which a diseased or otherwise damaged portion has been removed. Examples of other suitable grafts are described in U.S. Pat. Nos. 5,509,931, 5,527,353, and 5,556,426, all of which are herein incorporated by reference.

Examples of suitable stents include without limitation such as those described in U.S. Pat. No. 6,478,816 to Kveen et al., U.S. Pat. Nos. 4,655,771 and 4,954,126 issued to Wallsten and U.S. Pat. No. 5,061,275 issued to Wallsten et al. as well as U.S. Pat. No. 5,449,373 issued to Pinchasik et al.

The medical devices suitable for the invention may be fabricated from metallic, ceramic, polymeric materials, non-polymeric materials or combinations thereof. The material may be porous or nonporous. Porous structural elements can be microporous, nanoporous or mesoporous. Preferred materials are metallic. Suitable metallic materials include metals and alloys based on titanium (such as nitinol, nickel titanium alloys, thermo-memory alloy materials), stainless steel, tantalum, nickel-chrome, or certain cobalt alloys including cobalt-chromium-nickel alloys such as Elgiloy® and Phynox®. The components may also include parts made from other metals such as, for example, gold, platinum, or tungsten. The medical device may also include non-alloy combinations such as plated metals including but not limited to, for example, gold-plated or tantalum-plated stainless steel. Metallic materials also include clad composite filaments, such as those disclosed in WO 94/16646.

Suitable ceramic materials include, but are not limited to, oxides, carbides, or nitrides of the transition elements such as titaniumoxides, hafnium oxides, iridiumoxides, chromium oxides, aluminum oxides, and zirconiumoxides. Silicon based materials, such as silica, may also be used.

The polymer(s) useful for forming the medical devices should be ones that are biocompatible and avoid irritation to body tissue. The polymers can be either biostable or bioabsorbable. Suitable polymeric materials include without limitation polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, polystyrene, isobutylene, cellulose, collagens, and chitins.

Other polymers that are useful include, without limitation, dacron polyester, poly(ethylene terephthalate), polycarbonate, polymethylmethacrylate, polypropylene, polyalkylene oxalates, polyvinylchloride, polyurethanes, polysiloxanes, nylons, poly(dimethyl siloxane), polycyanoacrylates, polyphosphazenes, poly(amino acids), ethylene glycol I dimethacrylate, poly(methyl methacrylate), poly(2-hydroxyethyl methacrylate), polytetrafluoroethylene poly(HEMA), polyhydroxyalkanoates, polytetrafluorethylene, polycarbonate, poly(glycolide-lactide) co-polymer, polylactic acid, poly(γ-caprolactone), poly(γ-hydroxybutyrate), polydioxanone, poly(γ-ethyl glutamate), polyiminocarbonates, poly(ortho ester), polyanhydrides, alginate, dextran, chitin, cotton, polyglycolic acid, polyurethane, or derivatized versions thereof, i.e., polymers which have been modified to include, for example, attachment sites or cross-linking groups, e.g., RGD, in which the polymers retain their structural integrity while allowing for attachment of cells and molecules, such as proteins, nucleic acids, and the like. Furthermore, although the invention can be practiced by using a single type of polymer to form the medical device, various combinations of polymers can be employed. The appropriate mixture of polymers can be coordinated to produce desired effects when incorporated into a medical device.

Medical devices may be made with non-polymeric materials. Examples of useful non-polymeric materials include sterols such as cholesterol, stigmasterol, β-sitosterol, and estradiol; cholesteryl esters such as cholesteryl stearate; C₁₂ -C₂₄ fatty acids such as lauric acid, myristic acid, palmitic acid, stearic acid, arachidic acid, behenic acid, and lignoceric acid; C₁₈ -C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆ -C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; sphingomyelins such as stearyl, palmitoyl, and tricosanyl sphingomyelins; ceramides such as stearyl and palmitoyl ceramides; glycosphingolipids; lanolin and lanolin alcohols; and combinations and mixtures thereof. Preferred non-polymeric materials include cholesterol, glyceryl monostearate, glycerol tristearate, stearic acid, stearic anhydride, glyceryl monooleate, glyceryl monolinoleate, acetylated monoglycerides, fibrin, lactose, cellulose, polyvinylpyrrolidone, crospovidone, polyethylene glycol, and Enduragit™.

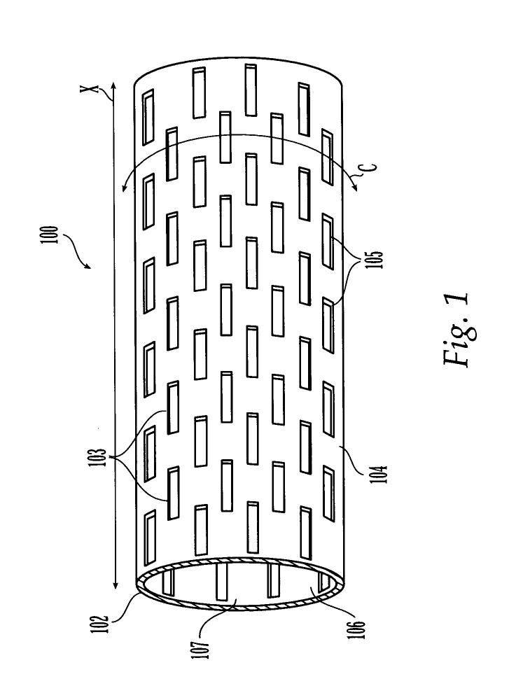

FIG. 1 shows an example of a stent 100 that can be used in the present invention. As discussed above, medical devices in addition to stents can be used in the present invention. The stent 100 comprises a tubular sidewall 102 comprising a plurality of struts 103. Also the stent 100 tubular sidewall 102 has an outer surface 104 having a plurality of openings 105 therein, an inner surface 106 having a plurality of openings 105 therein, and a flow path 107 that runs along the longitudinal axis X of the stent 100. The outer surface 104 is the surface facing away from the flow path 107 and the inner surface 106 is the surface facing the flow path 107. The stent 100 also has a circumference C.

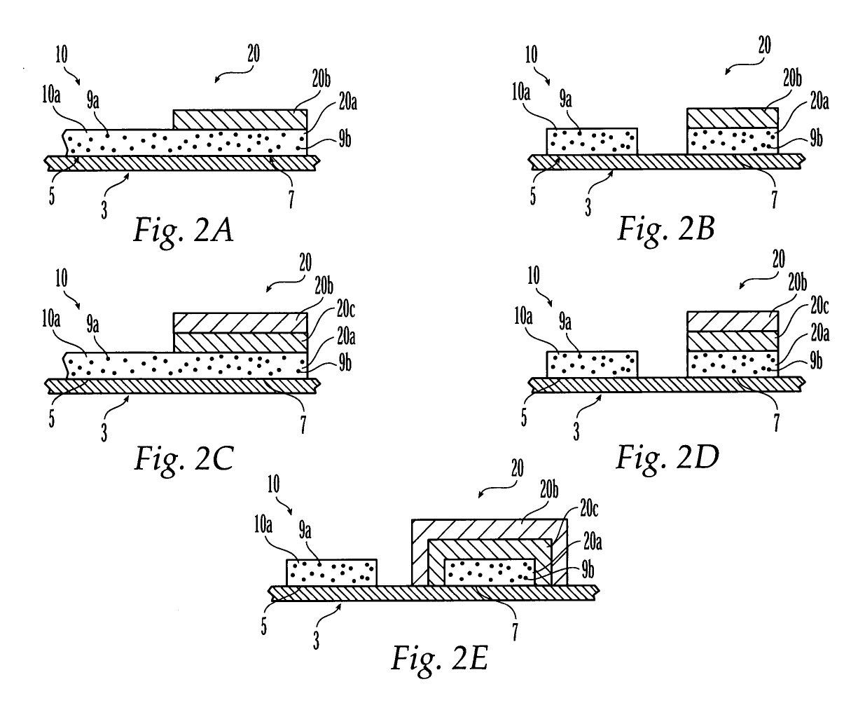

FIG. 2A shows an embodiment of the present invention. More specifically, this figure shows a cross-sectional view of a surface 3 of a medical device that is coated with a first coating region 10 and a second coating region 20. The first coating region 10 is disposed on a first portion of the surface 5 and the second coating region 20 is disposed on a second portion of the surface 7.

The first coating region 10 comprises a first coating layer 10a, which comprises a first therapeutic agent 9a. This first coating layer 10a can also comprise a polymeric material, such as those described below. In some embodiments, the polymeric material can incorporate the therapeutic agent. Although the first coating layer 10a is shown as being directly disposed on the first portion of the surface 5, i.e. in contact with the first portion of the surface 5, the first coating layer 10a can be indirectly disposed on the first portion of the surface 5. In such instance, an intermediate coating layer may be disposed between the first portion of the surface 5 and the first coating layer 10a. Also, as shown in FIG. 2A, in some embodiments, the first coating layer 10a not be covered by any other coating layer.

The second coating region 20 comprises a second coating layer 20a and a first additional coating layer 20b. The second coating layer 20a comprises a second therapeutic agent 9b, which could be the same as or different than the first therapeutic agent 9a. Also, the second coating layer 20a can include other therapeutic agents in addition to the second therapeutic agent 9b. Also, like the first coating layer 10a, the second coating layer 20a can also comprise a polymeric material. In some embodiments, the polymeric material can incorporate the therapeutic agent. Although the second coating layer 20a is shown as being directly disposed on the second portion of the surface 7, i.e. in contact with the second portion of the surface 7, the second coating layer 20a can be indirectly disposed on the second portion of the surface 7. For instance, an intermediate coating layer may be disposed between the second portion of the surface 7 and the second coating layer 20a.

The first additional coating layer 20b is disposed over the second coating layer 20a. The first additional coating layer 20b comprises a biodegradable material, such as a biodegradable polymer. Although not shown in FIG. 2A, the second coating region 20 can include other additional coating layers, such as a second additional coating layer disposed over the first additional coating layer 20b. Also, although FIG. 2a shows the first additional coating layer 20b covering the entire top surface of the second coating layer 20a, the first additional coating layer 20b or any other overlying layer may cover only a portion of the second coating layer 20a or other underlying coating layer.

The inclusion of the first additional coating layer 20b can prevent the therapeutic agent 9b of the second coating layer 20a from beginning to release from the second coating layer 20a at the same time that the therapeutic agent 9a of the first coating layer 10a begins to release from the first coating layer 10a. Since the second coating layer 20a is covered by the first additional coating layer 20b, in certain embodiments the second therapeutic agent 9b of the second coating layer 20a will not begin releasing from the second coating layer 20a generally until a significant part of the biodegradable first additional coating layer 20b has dissociated, i.e. degraded or absorbed. In contrast, because the first coating layer 10a is not covered by any additional coating layer, the first therapeutic agent 9a of the first coating layer 10a can begin to release from the first coating layer 10a without waiting for the degradation of an additional coating layer. Therefore, the inclusion of the first additional coating layer 20b can cause the therapeutic agents of the first and second coating layers, 10a and 20a, to be released over different time periods.

Also, in the embodiment shown in FIG. 2A, the first coating layer 10a and second coating layer 20a are contiguous, i.e. in contact with each other. In such an embodiment the first coating region 10 and second coating region 20 are in contact with each other. In other embodiments, such as that shown in FIG. 2B, the first and second coating regions can be spaced apart. Also, it should be noted that while FIGS. 2A-2B show only two coating regions, the surface 3 can be coated with additional coating regions.

FIGS. 2C-2D show embodiments of the coated medical device surface wherein the second coating region 20 comprises an intermediate layer 20c disposed between the second coating layer 20a and the first additional coating layer 20b. In these embodiments, the first additional coating layer 20b is indirectly disposed on or not in contact with the second coating layer 20a. In contrast, the first additional coating layer 20b is directly disposed on the second coating layer 20a in the embodiments of FIGS. 2A-2B. Preferably, the intermediate layer 20c comprises a biodegradable material that is the same or different as the biodegradable material of the first additional coating layer 20b. Also, the biodegradable material of the intermediate layer 20c can degrade at the same or a different rate than the biodegradable material of the first additional layer 20b. In some embodiments, the intermediate layer 20c can include a therapeutic agent.

In certain embodiments, such as that shown in FIG. 2E, the first additional coating layer 20b, or any other overlying layer, may not only cover the top surface of the second coating layer 20a or other underlying layer, such as an intermediate layer 20c, but also the side surfaces of the underlying layer. In this figure, the intermediate layer 20c covers the top and side surfaces of the second coating layer 20a. The first additional coating layer 20b covers the top and side surfaces of the intermediate layer 20c.

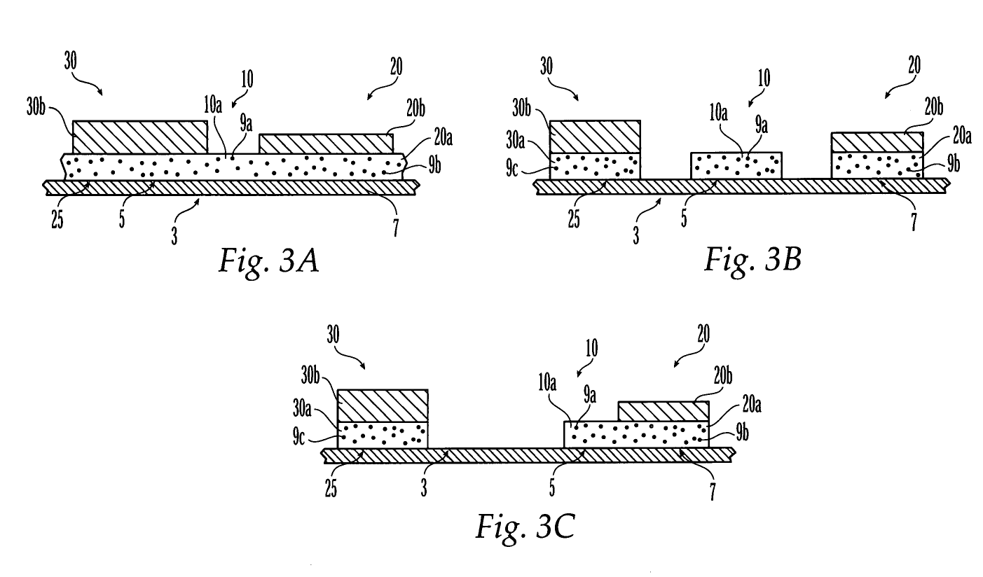

FIGS. 3A-3C show embodiments where the medical device surface 3 is coated with a third coating region 30 that is disposed over a third portion of the surface 25. The third coating region 30, comprises a third coating layer 30a and a second additional coating layer 30b.

The third coating layer 30a comprises a third therapeutic agent 9c that can be the same or different as the therapeutic agents 9a and 9b of the first and second coating layers, 10a and 20a respectively. Also, the third coating layer 30a can also include therapeutic agents in addition to the third therapeutic agent. Also, like the first and second coating layers 10a, 20a the third coating layer 30a can also comprise a polymeric material. In some embodiments, the polymeric material can incorporate the therapeutic agent. Although the third coating layer 20a is shown as being directly disposed on the third portion of the surface 25, i.e. in contact with the third portion of the surface 25 the third coating layer 30a can be indirectly disposed on the third portion of the surface 25. For instance, an intermediate coating layer may be disposed between the third portion of the surface 25 and the third coating layer 30a.

The second additional coating layer 30b is disposed over the third coating layer 30a. The second additional coating layer 30b comprises a biodegradable material, such as a biodegradable polymer. Although not shown in FIGS. 3A-3C, the third coating region 30 can include other additional coating layers, such as a third additional coating layer disposed over the second additional coating layer 30b. Moreover, the first and second additional coating layers, 20b and 30b can have the same or different thicknesses.

Like the first additional coating layer 20b, the inclusion of the second additional coating layer 30b affects the time when the therapeutic agent of its underlying coating layer(s) begins to release from the underlying coating layers, e.g., third coating layer 30a. Therefore, inclusion of this second additional coating layer 30b can result in a third coating region 30 that releases its therapeutic agent over a time period that is different than the time period(s) over when at least one other coating region releases its therapeutic agent.

In the embodiment shown in FIG. 3A, the first coating layer 10a is contiguous with the second coating layer 20a which is contiguous with the third coating layer 30a. In other embodiments, such as that shown in FIG. 3B, the first, second and third coating regions are spaced apart. Alternatively, as shown in FIG. 3C, the first and second coating regions 10, 20, can be contiguous while the third coating region 30 is spaced apart from the first and second coating regions 10, 20. Also, it should be noted that while FIGS. 3A-3C show only three coating regions, the surface 3 can be coated with additional coating regions.

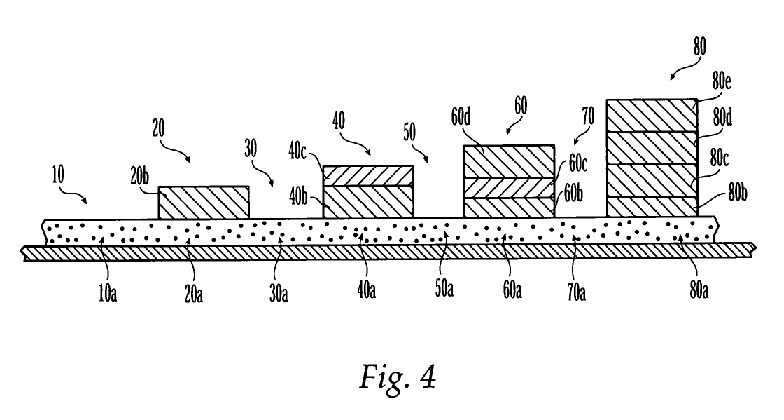

FIG. 4 shows an embodiment where the medical device surface is coated with 8 coating regions, 10, 20, 30, 40, 50, 60, 70 and 80. The first coating region 10, the third coating region 30, the fifth coating region 50 and the seventh coating region 70 each comprise a single coating layer, 10a, 30a, 50a and 70a respectively. These coating layers may comprise a therapeutic agent, which can be the same or different in each of the coating layers. In other embodiments, these coating layers may be free of any therapeutic agent. The coating layers 10a, 30a, 50a, and 70a, are not covered by any other coating layer.

The second, fourth, sixth and eighth coating regions 20, 40, 60 and 80 include at least one biodegradable coating layer overlying a coating layer 20a, 40a, 60a and 80a that comprises a therapeutic agent. The second coating region 20 comprises one additional coating layer 20b that overlies coating layer 20a, which includes a therapeutic agent. Preferably, the biodegradable additional coating layer 20b comprises a biodegradable polymeric material.

The fourth coating region 40 comprises two additional biodegradable coating layers 40b, 40c that overlie coating layer 40a, which includes a therapeutic agent. Preferably, the biodegradable additional coating layers 40b, 40c comprise a biodegradable polymeric material. Alternatively, either or both additional coating layers 40b, 40c can comprise a therapeutic agent and/or a biodegradable polymeric material.

The sixth coating region 60 comprises three additional biodegradable coating layers 60b, 60c and 60d that overlie coating layer 60a, which includes a therapeutic agent. Preferably, the biodegradable additional coating layers 60b, 60c and 60d comprise a biodegradable polymeric material. Alternatively, any of the additional coating layers 60b, 60c and 60d can comprise a therapeutic agent and/or a biodegradable polymeric material.

The eighth coating region 80 comprises four additional biodegradable coating layers 80b, 80c, 80d and 80e that overlie coating layer 80a, which includes a therapeutic agent. Preferably, the biodegradable additional coating layers 80b, 80c, 80d and 80e comprise a biodegradable polymeric material. Alternatively, any of the additional coating layers 80b, 80c, 80d and 80e can comprise a therapeutic agent and/or a biodegradable polymeric material. For example, additional coating layers 80b and 80c may contain therapeutic agents and a biodegradable polymeric material while additional coating layers 80d and 80e contain a biodegradable polymeric material without any therapeutic agent.

Since the coating regions shown in FIG. 4, comprise different numbers of additional coating layers comprising various coating materials, the time when the therapeutic agent of the underlying coating layers 10a, 20a, 30a, 40a, 50a, 60a, 70a and 80a begins to be released from the each underlying coating layer can vary. Therefore, in this embodiment the time periods when the therapeutic agent(s) of the underlying coating layers 10a, 20a, 30a, 40a, 50a, 60a, 70a and 80a are release may generally vary from coating region to coating region.

In some embodiments, the coating region can begin to deliver the therapeutic agent immediately after the medical device is implanted. In specific embodiments, the coating region can begin to deliver the therapeutic agent about 10 to 60 minutes, 1-3 hours, 3-5 hours, 5-12 hours, 12-24 hours, 1-2 days, 2-7 days, 1-2 weeks, 2-4 weeks, 1-3 months, 3-6 months, 6-9 months, 9-12 months, 1-2 years, 2-4 years, 4-6 years, or 6-10 years after implantation of the medical device. In certain embodiments, the coating region can release the therapeutic agent for a period of at least about 10-60 minutes, 1-3 hours, 3-5 hours, 5-12 hours, 12-24 hours, 1-2 days, 2-7 days, 1-2 weeks, 2-4 weeks, 1-3 months, 3-6 months, 6-9 months, 9-12 months, 1-2 years, 2-4 years, 4-6 years, or 6-10 years. However, depending on the properties of the biodegradable/bioabsorbable layers, the actual therapeutic release time may be longer or shorter.

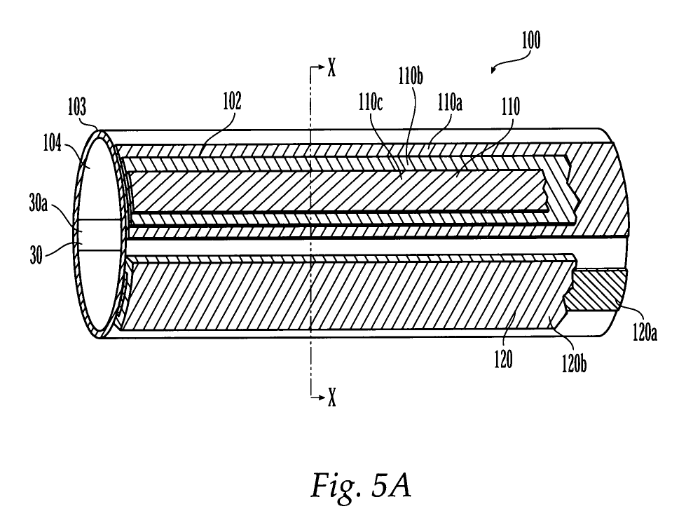

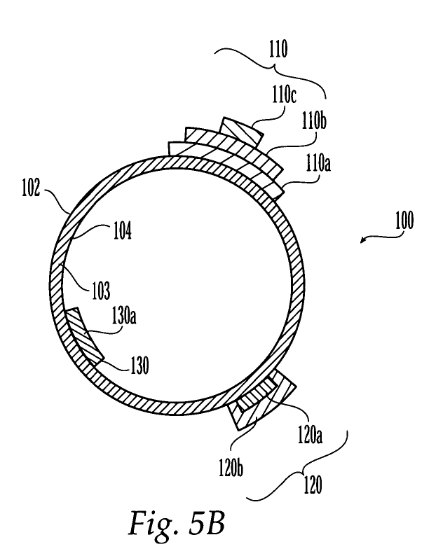

The coating region can be disposed on the surface of the medical device in any desired configuration or shape, e.g. circle, rectangle, etc. or may even be disposed in a pattern. For example, when the medical device is a stent, the coating regions can be disposed along the longitudinal axis of the stent as shown in FIG. 5A. FIG. 5B shows a cross-sectional view of the stent of FIG. 5A along line X-X. In this embodiment, there are two coating regions 110 and 120 disposed on the outer surface of the stent and a third coating region 130 disposed on the inner surface of the stent. In some embodiments the coating regions are disposed on either the outer surface and/or the inner surface.

FIG. 5A shows a stent 100 having two coating regions, a first coating region 110 and a second coating region 120, disposed on the outer surface 102 of its sidewall 103 as well as a third coating region 130 disposed on the inner 104 surface of the sidewall 103. Although not shown, the side wall 103 can include a plurality of openings that extend through the outer surface 102 and inner surface 104. As shown in the cut-away section of the first coating region 110 in FIG. 5A, this coating region comprises three coating layers: a first coating layer 110a, a first additional coating layer 110b and a second additional coating layer 110c. In this embodiment the second additional coating layer 110c does not cover the entire first additional coating layer 110b, which in turn does not cover the entire first coating layer 110a. FIG. 5B shows a cross-sectional view of this coating region. In other embodiments this region can include further coating layers.

The second coating region 120 comprises a second coating layer 120a and a third additional layer 120b. In this embodiment, as shown in the cut-away section of FIG. 5A and the cross-section view of FIG. 5B, the overlying third additional coating layer 120b covers the entire underlying second coating layer 120a. In certain embodiments, the overlying coating layers do not have to cover the entire underlying coating layer, e.g., cover just the top surface of the underlying layer. The third coating region 130 comprises a single third coating layer 130a which is not covered by another coating layer. In some embodiments the third coating region 130 can include additional coating layers. Although the coating regions are shown as extending the entire length of the stent, in some embodiments, some or all of the coating regions may only extend a part of the length of the stent. Also, in some embodiments there may be more or fewer coating regions, having various numbers of layers.

In some embodiments, the first, second and/or third coating layers, 110a, 120a and 130a comprise a therapeutic agent. In certain embodiments, the first, second and/or third additional coating layers, 110b, 120b and 110a comprise a biodegradable material and/or a therapeutic agent.

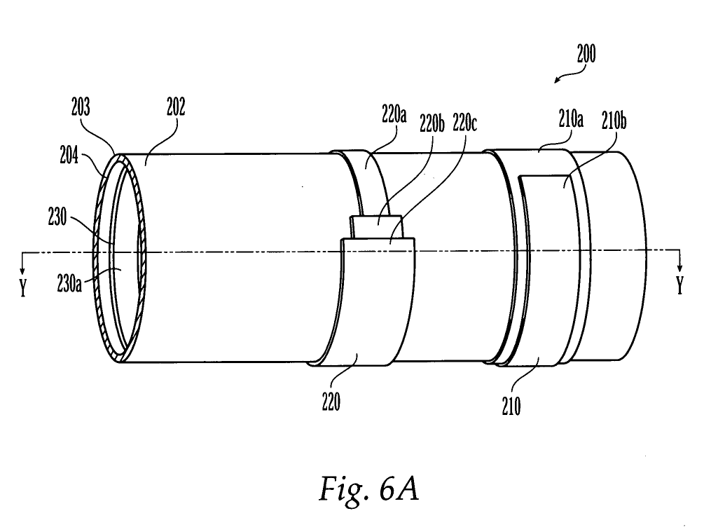

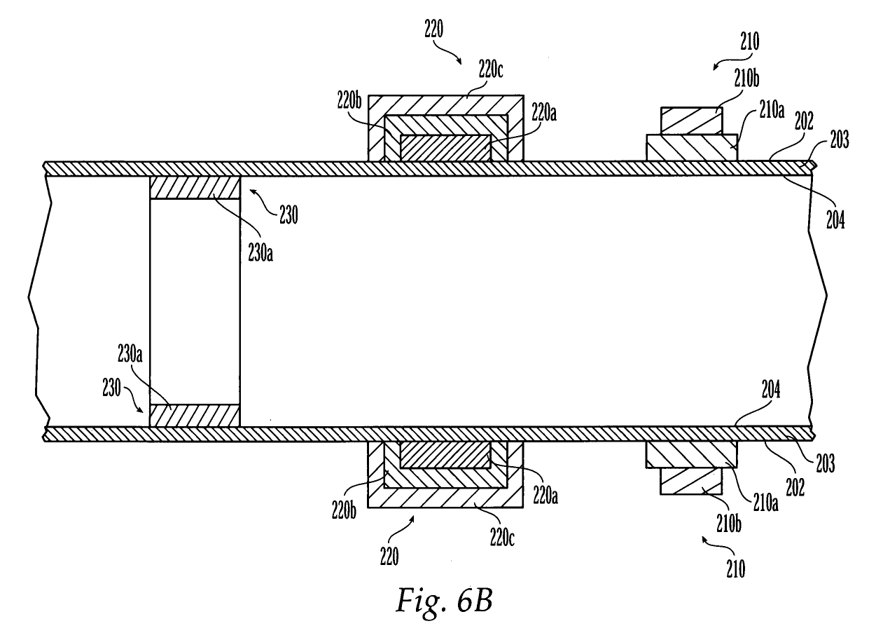

Alternatively, the coating regions can be disposed along the circumference of the stent as shown as FIGS. 6A-6B. As shown in FIGS. 6A-6B, in this embodiment, there are three coating regions 210, 220 and 230. The first coating region 210 and second coating region 220 are disposed on the outer surface 202 of the sidewall 203 of the stent 200. The third coating region 230 is disposed on the inner surface 204 of the sidewall 203 of the stent 200. In other embodiments, there can be more or fewer coating regions. Also, in other embodiments, the coating regions can have different number and types of coating layers than what are shown in the figures. Furthermore, the sidewall 203 can include openings (not shown) that extend through the inner 204 and outer 202 surfaces. Moreover, although the coating regions are shown to span an entire circumference of the stent, in some embodiments, less than the entire circumference may be covered by a coating region.

The first coating region 210 comprises two coating layers: a first coating layer 210a and a first additional coating layer 210b. As shown in the cut-away portion of FIG. 6A and the cross-sectional view of FIG. 6B, the first additional layer 210b does not cover the underlying first coating layer 210a. In other embodiments, the first additional coating layer 210a can cover the entire first coating layer 210a or if the first coating region includes other coating layers, those other coating layers.

The second coating region 220 comprises three coating layers in this embodiment; a second coating layer 220a, a second additional coating layer 220b and a third additional coating layer 220c. In this embodiment, the third additional coating layer 220c covers the entire underlying second additional coating layer 220b, which covers the entire underlying second coating layer 220a. (See cut-away in FIG. 6A and the cross-sectional view of FIG. 6B). In other embodiments, the overlying coating layer may cover less than all of an underlying coating layer. Also in other embodiments the third coating region 230 can include more or fewer coating layers.

The third coating region 230 comprises a third coating layer 230a. In this embodiment, the third coating layer 230a, is not covered by any other coating layer. In other embodiments it may be covered by other coating layer(s).

In some embodiments, the first, second and/or third coating layers 210a, 220a, and 230a comprise a therapeutic agent. In certain embodiments, the first, second and/or third additional coating layers 220b, 220c and 210b comprise a biodegradable material and/or a therapeutic agent.

The various coating layers of the coating regions can be formed by applying a coating composition to the medical device. Coating compositions may be applied by any method to a surface of a stent or medical device to form a coating layer. Examples of suitable methods include, but are not limited to, spraying such as by conventional nozzle or ultrasonic nozzle, dipping, rolling, electrostatic deposition, and a batch process such as air suspension, pancoating or ultrasonic mist spraying. Other coating methods include screen printing, positive displacement coating (i.e., inkjet-printing technology), and spray coating combined with masking. Also, more than one coating method may be used.

Coating compositions for forming coating layers may include a polymeric material. The polymeric material should be a material that is biocompatible and avoids irritation to body tissue. Preferably the polymeric materials used in the coating composition of the present invention are selected from the following: polyurethanes, silicones (e.g., polysiloxanes and substituted polysiloxanes), and polyesters. Also preferable as a polymeric material are styrene-isobutylene-styrene copolymers. Other polymers that may be used include ones that may be dissolved and cured or polymerized on the stent or polymers having relatively low melting points that can be blended with biologically active materials. Additional suitable polymers include thermoplastic elastomers in general, polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, polyethers, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, EPDM (ethylene-propylene-diene) rubbers, fluorosilicones, polyethylene glycol, polysaccharides, phospholipids, and combinations of the foregoing.

Preferably, for medical devices which undergo mechanical challenges, e.g., expansion and contraction, polymeric materials should be selected from elastomeric polymers such as silicones (e.g., polysiloxanes and substituted polysiloxanes), polyurethanes, thermoplastic elastomers, ethylene vinyl acetate copolymers, polyolefin elastomers, and EPDM rubbers. Because of the elastic nature of these polymers, the coating composition is capable of undergoing deformation under the yield point when the stent is subjected to forces, stress or mechanical challenge.

The biodegradable polymers that can be used to form the biodegradable coating layers include without limitation poly-L-lactic acid, poly-glycolic acid, polycaprolactone, poly(lactide-co-glycolide), poly(hydroxybutyrate), poly(hydroxybutyrate-co-valerate), polydioxanone, polyorthoester, polyanhydride, poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(etheresters)(e.g., PEO/PLA), polyalkylene oxalates, polyphosphazenes and biomolecules such as fibrin, fibrinogen, cellulose, starch, collagen, gel foam and hyaluronic acid.

The therapeutic agents that can be included in the coating layers include biologically active agents, and also genetic materials and pharmaceutical or nutraceutical materials. The genetic materials mean DNA or RNA, including, without limitation, of DNA/RNA encoding a useful protein stated below, intended to be inserted into a human body including viral vectors and non-viral vectors as well as anti-sense nucleic acid molecules such as DNA, RNA and RNAi. Viral vectors include adenoviruses, gutted adenoviruses, adeno-associated virus, retroviruses, alpha virus (Semliki Forest, Sindbis, etc.), lentiviruses, herpes simplex virus, ex vivo modified cells (e.g., stem cells, fibroblasts, myoblasts, satellite cells, pericytes, cardiomyocytes, skeletal myocytes, macrophage), replication competent viruses (e.g., ONYX-015), and hybrid vectors. Non-viral vectors include artificial chromosomes and mini-chromosomes, plasmid DNA vectors (e.g., pCOR), cationic polymers (e.g., polyethyleneimine, polyethyleneimine (PEI)) graft copolymers (e.g., polyether-PEI and polyethylene oxide-PEI), neutral polymers PVP, SP1017 (SUPRATEK), lipids or lipoplexes, nanoparticles and microparticles with and without targeting sequences such as the protein transduction domain (PTD). The biological materials include cells, yeasts, bacteria, proteins, peptides, cytokines and hormones. Examples for peptides and proteins include growth factors (FGF, FGF-1, FGF-2, VEGF, Endothelial Mitogenic Growth Factors, and epidermal growth factors, transforming growth factor and platelet derived endothelial growth factor, platelet derived growth factor, tumor necrosis factor, hepatocyte growth factor and insulin like growth factor), transcription factors, proteinkinases, CD inhibitors, thymidine kinase, and bone morphogenic proteins (BMP's), such as BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells may be of human origin (autologous or allogeneic) or from an animal source (xenogeneic), genetically engineered, if desired, to deliver proteins of interest at the transplant site. The delivery media can be formulated as needed to maintain cell function and viability. Cells include whole bone marrow, bone marrow derived mono-nuclear cells, progenitor cells (e.g., endothelial progentitor cells) stem cells (e.g., mesenchymal, hematopoietic, neuronal), pluripotent stem cells, fibroblasts, macrophage, and satellite cells.

Therapeutic agents also include non-genetic therapeutic agents, such as:

anti-thrombogenic agents such as heparin, heparin derivatives, urokinase, and PPack (dextrophenylalanine proline arginine chloromethylketone);

anti-proliferative agents such as enoxaprin, angiopeptin, or monoclonal antibodies capable of blocking smooth muscle cell proliferation, hirudin, acetylsalicylic acid, tacrolimus, everolimus, amlodipine and doxazosin;

anti-inflammatory agents such as glucocorticoids, betamethasone, dexamethasone, prednisolone, corticosterone, budesonide, estrogen, sulfasalazine, rosiglitazone, mycophenolic acid, and mesalamine;

antineoplastic/antiproliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxi and its analogs or derivatives;

anesthetic agents such as lidocaine, bupivacaine, and ropivacaine;

anti-coagulants such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory biologically active agent), dipyridamole, protamine, hirudin, prostaglandin inhibitors, platelet inhibitors, antiplatelet agents such as trapidil and liprostin, and tick antiplatelet peptides;

DNA demethylating drugs such as 5-azacytidine, which is also categorized as a RNA or DNA metabolite that inhibit cell growth and induce apoptosis in certain cancer cells.

vascular cell growth promotors such as growth factors, vascular Endothelial Growth Factors (FEGF, all types including VEGF-2), growth factor receptors, transcriptional activators, and translational promotors;

vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;

cholesterol-lowering agents; vasodilating agents; and agents which interfere with endogenous vasoactive mechanisms;

anti-oxidants, such as probucol;

antibiotic agents, such as penicillin, cefoxitin, oxacillin, tobranycin, rapamycin (sirolimus);

angiogenic substances, such as acidic and basic fibrobrast growth factors, estrogen including estradiol (E2), estriol (E3) and 17-Beta Estradiol; and

biologically active agents for heart failure, such as digoxin, beta-blockers, angiotensin-converting enzyme (ACE) inhibitors including captopril and enalopril, statins and related compounds.

Preferred therapeutic agents include anti-proliferative drugs such as steroids, vitamins, and restenosis-inhibiting agents. Preferred restenosis-inhibiting agents include microtubule stabilizing agents such as Taxol, paclitaxel, paclitaxel analogues, derivatives, and mixtures thereof. For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl)glutamine, and 2′-O-ester with N-(dimethylaminoethyl) glutamide hydrochloride salt.

Other preferred therapeutic agents include nitroglycerin, nitrous oxides, nitric oxides, antibiotics, aspirins, digitalis, estrogen derivatives such as estradiol and glycosides.

Coating compositions suitable for applying therapeutic agents to the devices of the present invention preferably include a polymeric material and a therapeutic agent dispersed or dissolved in a solvent which does not alter or adversely impact the therapeutic properties of the biologically active material employed. Suitable polymers and therapeutics include, but are not limited to, those listed above.

Solvents used to prepare coating compositions include ones which can dissolve or suspend the polymeric material in solution. Examples of suitable solvents include, but are not limited to, tetrahydrofuran, methylethylketone, chloroform, toluene, acetone, isooctane, 1,1,1,-trichloroethane, dichloromethane, isopropanol, IPA, and mixtures thereof.

In another embodiment, the coating composition comprises a non-polymeric material. In another embodiment, the coating composition comprises entirely of a therapeutic agent. Coating compositions may be used to apply one type of therapeutic agent or a combination of therapeutic agents.

It should be appreciated that the features and components described herein may be used singly or in any combination thereof. Moreover, the present invention is not limited to only the embodiments specifically described herein, and may be used with medical devices other than stents. The disclosed system may be used to deliver a therapeutic agent to various types of body lumina, including but not limited to the esophagus, urinary tract, and intestines. The description contained herein is for purposes of illustration and not for purposes of limitation. Changes and modifications may be made to the embodiments of the description and still be within the scope of the invention. Furthermore, obvious changes, modifications or variations will occur to those skilled in the art. Also, all references cited above are incorporated herein, in their entirety, for all purposes related to this disclosure.

While the foregoing description and drawings may represent preferred embodiments of the present invention, it should be understood that various additions, modifications, and substitutions may be made therein without departing from the spirit and scope of the present invention as defined in the accompanying claims. In particular, it will be clear to those skilled in the art that the present invention may be embodied in other specific forms, structures, arrangements, and proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being indicated by the appended claims and not limited to the foregoing description.

What is claimed is:

1. A medical device for delivering a therapeutic agent to a body tissue of a patient comprising:
(a) a medical device having a surface;
(b) a first coating region disposed on a first portion of the medical device surface, wherein the first coating region comprises a first coating layer comprising a first therapeutic agent;
(c) a second coating region disposed on a second portion of the medical device surface, wherein the second coating region comprises
(i) a second coating layer comprising a second therapeutic agent; and
(ii) at least a first additional coating layer disposed over the second coating layer, wherein the first additional coating layer comprises a first biodegradable or bioabsorbable material,
and wherein the first coating region is capable of releasing the first therapeutic agent before the second coating region begins to release the second therapeutic agent.

2. The medical device of claim 1 wherein the second therapeutic agent begins to release before the release of the first therapeutic agent is completed.

3. The medical device of claim 1 wherein the second therapeutic agent begins to release after the release of the first therapeutic agent is completed.

4. The medical device of claim 1, wherein the first and second therapeutic agents are the same.

5. The medical device of claim 1, wherein the first coating layer is not covered by any other coating layer.

6. The medical device of claim 1, wherein the first coating layer and the second coating layer are contiguous.

7. The medical device of claim 1, wherein the first additional coating layer is disposed directly over the second coating layer.

8. The medical device of claim 1, wherein the first additional coating layer is disposed indirectly over the second coating layer.

9. The medical device of claim 8, further comprising an intermediate coating layer disposed between the second coating layer and the first additional coating layer.

10. The medical device of claim 9, wherein the intermediate coating layer comprises a biodegradable or bioabsorbable material.

11. The medical device of claim 9, wherein the intermediate coating layer comprises a third therapeutic agent.

12. The medical device of claim 11, wherein the intermediate coating layer further comprises a biodegradable or bioabsorbable material.

13. The medical device of claim 1, wherein the first coating layer further comprises a polymeric material.

14. The medical device of claim 1, wherein the second coating layer further comprises a polymeric material.

15. The medical device of claim 1, wherein the first therapeutic agent comprises an anti-thrombogenic agent, an anti-angiogenesis agent, an anti-proliferative agent, a growth factor, or a radiochemical.

16. The medical device of claim 15, wherein the anti-proliferative agent comprises paclitaxel, a paclitaxel analogue or a paclitaxel derivative.

17. The medical device of claim 1, wherein the medical device is a stent having a tubular sidewall and wherein the first portion of the medical device surface lies along a circumference of the tubular sidewall.

18. The medical device of claim 1, wherein the medical device is a stent having a tubular sidewall and wherein the first portion of the medical device surface lies along a longitudinal axis of the tubular sidewall.

19. The medical device of claim 1, further comprising a third coating region disposed over a third portion of the medical device surface wherein the third coating region comprises
(i) a third coating layer comprising a third therapeutic agent; and
(ii) at least a second additional coating layer disposed over the third coating layer, wherein the second additional coating layer comprises a second biodegradable or bioabsorbable material.

20. The medical device of claim 19, wherein the first, second and third therapeutic agent are the same.

21. The medical device of claim 19, wherein the second additional coating layer is disposed directly over the third coating layer.

22. The medical device of claim 19, wherein the second additional coating layer is disposed indirectly over the third coating layer.

23. The medical device of claim 22, further comprising an intermediate coating layer disposed between the second coating layer and the first additional coating layer.

24. The medical device of claim 23, wherein the intermediate coating layer comprises a biodegradable or bioabsorbable material.

25. The medical device of claim 23, wherein the intermediate coating layer comprises a fourth therapeutic agent.

26. The medical device of claim 25, wherein the intermediate coating layer further comprises a biodegradable or bioabsorbable material.

27. The medical device of claim 19, wherein the first coating layer, is contiguous with at least the second coating layer or the third coating layer.

28. The medical device of claim 19, further comprising at least a third additional coating layer disposed over the second additional coating layer.

29. The medical device of claim 28, wherein the third additional coating layer comprises a third biodegradable or bioabsorbable material.

30. The medical device of claim 19, wherein the first and second biodegradable or bioabsorbable materials degrade at the same rate.

31. The medical device of claim 19, wherein the first and second biodegradable or bioabsorbable materials degrade at different rates.

32. The medical device of claim 19, wherein the first additional coating layer has a first thickness and the second additional coating layer has a second thickness and wherein the first and second thicknesses are not the same.

33. The medical device of claim 1, wherein the medical device is a stent having a tubular sidewall having an inner surface and an outer surface and wherein the first coating region and second coating region are disposed on the outer surface.

34. The medical device of claim 33 further comprising a third coating region disposed on the inner surface, wherein the third coating region comprises
(i) a third coating layer comprising a third therapeutic agent; and
(ii) at least a second additional coating layer disposed over the third coating layer, wherein the second additional coating layer comprises a second biodegradable or bioabsorbable material.

35. A stent for delivering a therapeutic agent to patient comprising:
(a) a surface;
(b) a first coating region disposed on a first portion of the surface, wherein the first coating region comprises a first coating layer comprising a first therapeutic agent and a first polymeric material;
(c) a second coating region disposed on a second portion of the medical device surface, wherein the second coating region comprises
(i) a second coating layer comprising a second therapeutic agent and a second polymeric material; and
(ii) at least an additional coating layer disposed over the second coating layer, wherein the additional coating layer comprises a first biodegradable or bioabsorbable material,
and wherein the first coating region is capable of releasing the first therapeutic agent before the second coating region begins to release the second therapeutic agent, and
wherein the first coating layer and the second coating layer are contiguous and wherein the additional coating layer is disposed directly over the second coating layer.

36. The medical device of claim 35 wherein the second therapeutic agent begins to release before the release of the first therapeutic agent is completed.

37. The medical device of claim 35 wherein the second therapeutic agent begins to release after the release of the first therapeutic agent is completed.

38. A stent for delivering a therapeutic agent to a patient comprising:
(a) a tubular sidewall having an outer surface and an inner surface;
(b) a first coating region disposed on a first portion of the outer surface, wherein the first coating region comprises a first coating layer comprising a first therapeutic agent and a polymeric material;
(c) a second coating region disposed on a second portion of the outer surface, wherein the second coating region comprises
(i) a second coating layer comprising a second therapeutic agent and the polymeric material; and
(ii) at least a first additional coating layer disposed over the second coating layer, wherein the first additional coating layer comprises a first biodegradable or bioabsorbable material,
and wherein the first coating region is capable of releasing the first therapeutic agent before the second coating region begins to release the second therapeutic agent; and
(d) a third coating region disposed on a portion of the inner surface comprising
(i) a third coating layer comprising a third therapeutic agent and the polymeric material; and
(ii) at least a second additional coating layer disposed over the third coating layer, wherein the second additional coating layer comprises a second biodegradable or bioabsorbable material,
wherein the first coating layer, and the second coating layer are contiguous, and wherein the first additional coating layer is disposed directly over the second coating layer.

39. The stent of claim 38 wherein the second therapeutic agent begins to release before the release of the first therapeutic agent is completed.

40. The stent of claim 38 wherein the second therapeutic agent begins to release after the release of the first therapeutic agent is completed.

41. The medical device of claim 38 further comprising at least a third additional coating layer disposed over the second additional coating layer.

42. The medical device of claim 41, wherein the third additional coating layer comprises a third biodegradable or bioabsorbable material.

43. The medical device of claim 41 wherein the second additional coating layer further comprises a fourth therapeutic agent.

44. The medical device of claim 42 wherein the third additional coating layer further comprises a fifth therapeutic agent.
